(19) United States
(12) Patent Application Publication
(10) Pub. No.: US 2022/0323010 A1
(43) Pub. Date: Oct. 13, 2022

(54) PHYSIOLOGICAL SIGNAL MONITORING DEVICE AND MOUNTING METHOD THEREFOR

(71) Applicant: BIONIME CORPORATION, Taichung City (TW)

(72) Inventors: Chun-Mu Huang, Taichung City (TW); Chieh-Hsing Chen, Taichung City (TW); Chen-Hao Lee, Taichung City (TW)

(21) Appl. No.: 17/809,411

(22) Filed: Jun. 28, 2022

Related U.S. Application Data
(63) Continuation-in-part of application No. 16/944,621, filed on Jul. 31, 2020.
Application No. 16/944,621 is a continuation-in-part of application No. 16/944,783, filed on Jul. 31, 2020.
(60) Provisional application No. 62/882,140, filed on Aug. 2, 2019.
(60) Provisional application No. 62/882,140, filed on Aug. 2, 2019.

(30) Foreign Application Priority Data
Jan. 10, 2020 (TW) 109100852
Jan. 10, 2020 (TW) 109100968

(51) Int. Cl. A61B 5/00; A61B 5/145

(52) U.S. Cl. CPC A61B 5/6833; A61B 5/14532; A61B 2562/0295; A61B 2560/0412

(57) ABSTRACT

A physiological signal monitoring device includes a base, a biosensor and a transmitter. The biosensor includes a sensing section. The transmitter includes a connection part. The connection part and the signal output section cooperatively form a connection portion after the transmitter moving from an initial position to an assembled position. A safety gap is formed between the transmitter and the biosensor. When the transmitter moves from the initial position toward the assembled position, the safety gap serves to prevent at least one of the connection part of the transmitter and the signal output section of the biosensor from collision.

### CROSS-REFERENCE TO RELATED APPLICATION

This is a continuation-in-part application of U.S. patent application Ser. No. 16/944,621, filed on Jul. 31, 2020, which claims priority to U.S. Provisional Patent Application No. 62/882,140, filed on Aug. 2, 2019, and priority of Taiwanese Invention Patent Application No. 109100968, filed on Jan. 10, 2020, and a continuation-in-part application of U.S. patent application Ser. No. 16/944,783, filed on Jul. 31, 2020, which claims priority to U.S. Provisional Patent Application No. 62/882,140, filed on Aug. 2, 2019, and Taiwanese Patent Application No. 109100852, filed on Jan. 10, 2020.

### FIELD

The disclosure relates to a monitoring device, and more particularly to a physiological signal monitoring device and a mounting method therefor.

### BACKGROUND

In order to control diabetes or reduce its complications, control of blood glucose is very important. Regular blood glucose measurement and track the trend of blood glucose concentration changes can ensure that the blood glucose concentration remains at a safe and stable level in the long term. For the measurement of blood glucose concentration, a conventional method is to pierce the skin of a patient with a lancing device to collect blood, and then use the testing strip with the glucose meter to measure and calculate the glucose concentration in the collected blood by electrochemical analysis. However, the need of repeatedly collect and test the blood often causes inconvenience to the patient. Therefore, in the past two decades, a continuous glucose monitoring (CGM) system that can be at least partially implanted in the body to continuously monitor the real-time glucose level has been developed rapidly.

The continuous glucose monitoring system must be worn by a user for a long term, so miniaturization of the size thereof is needed. The basic structure of the continuous glucose monitoring system includes at least a sensor for measuring a physiological signal corresponding to the glucose level in the body and a transmitter for receiving and transmitting the physiological signal.

Referring to FIG. 19, a conventional sensing device 900 disclosed in U.S. Pat. No. 7,899,511 includes a mounting unit 92, an adhesive base 91 that is adapted for adhering the mounting unit 92 onto a host's skin (not shown), a biosensor 93 that mounted in the mounting unit 92, and a transmitter 94 that is mounted to the mounting unit 92 and that is connected to the biosensor 93. The biosensor 93 is inserted beneath the host's skin for measuring a physiological signal corresponding to the glucose level, and the transmitter 94 receives the physiological signal from the biosensor 93 and transmits the physiological signal to an external device, such as a receiver (not shown in the figure).

Due to the intrusive nature of the sensing device 900, the host's body may become hypersensitive to the biosensor 93, and in turn develops a severe allergic reaction. As such, the biosensor 93 has to be replaced on a weekly or hi-weekly basis. In comparison, as the transmitter 94 is relatively expensive, when the biosensor 93 is to be replaced, the transmitter 94 is usually disengaged from the mounting unit 92 for next uses. During the installation of the transmitter 94 onto the mounting unit 92, the biosensor 93 may be damaged due to collision with other components.

### SUMMARY

Therefore, an object of the disclosure is to provide a physiological signal monitoring device that can alleviate at least one of the drawbacks of the prior art.

According to the disclosure, the physiological signal monitoring device is adapted to be mounted to a skin surface of a host and to be partially inserted underneath the skin surface for measuring at least one analyte of the host, and includes a base, a biosensor and a transmitter. The base includes at least one first alignment structure that is disposed on a top portion of the base. The biosensor includes a sensing section that is adapted to be inserted underneath the skin surface for measuring the at least one analyte, and a signal output section that is disposed at the top portion of the base. The transmitter is selectively located at one of an initial position and an assembled position relative to the base in the direction of a first axis, and includes a connection part and at least one second alignment structure. The connection part is disposed at a bottom portion of the transmitter. The connection part and the signal output section of the biosensor are shaped to be complementary to each other, and cooperatively form a connection portion after the transmitter moving from the initial position to the assembled posit non by a first travel distance in the direction of the first axis. The second alignment structure is disposed at the bottom portion of the transmitter and correspond in position to the first alignment structure of the base. The second alignment structure and the first alignment structure are shaped to be complementary to each other, and cooperatively form an alignment portion after the transmitter moving from the initial position to the assembled position by a second travel distance in the direction of the first axis. The first travel distance is greater than or equal to the second travel distance, and a safety gap is formed between the transmitter and the biosensor. When the transmitter moves from the position toward the assembled position, the safety gap serves to prevent at least one of the connection part of the transmitter and the signal output section of the biosensor from collision.

Another object of the disclosure is to provide a method for mounting a physiological signal monitoring device onto the skin surface of the host that can alleviate at least one of the drawbacks of the prior art.

According to the disclosure, the method includes steps of: a) providing the physiological signal monitoring device in the above; b) executing a first transmitter mounting process, in which the transmitter is moved relative to the base in the direction of the first axis from the initial position toward the assembled position with the bottom portion of the transmitter facing the top portion of the base, and the safety gap prevent at least one of the signal output section of the biosensor and the connection part from collision; executing an alignment process, in which the relative movement between the first alignment structure and the second alignment structure during the first alignment structure and the second alignment structure are limited with each other guides movement between the transmitter and the base for aligning the signal output section of the biosensor and the connection part of the transmitter with each other; and d) executing a second transmitter mounting process, in which the transmitter is moved relative to the base in the direction of the first axis to be mounted onto the base, and the signal output section of the biosensor is connected to the connection part to form an electric connection between the biosensor and the transmitter.

### BRIEF DESCRIPTION OF THE DRAWINGS

Other features and advantages of the disclosure will become apparent in the following detailed description of the embodiments with reference to the accompanying drawings, of which:

FIG. 1 is a perspective view illustrating a first embodiment of the physiological signal monitoring device according to the disclosure;

FIG. 2 is a partly exploded perspective view illustrating the first embodiment;

FIG. 3 is another partly exploded perspective view illustrating the first embodiment;

FIG. 4 is a sectional view illustrating a transmitter of the first embodiment at an initial position;

FIG. 5 is a sectional view illustrating the transmitter at an assembled position;

FIG. 6 is another sectional view taken along line VI-VI in FIG. 5;

FIG. 7 is a sectional view illustrating a base and a biosensor of the first embodiment being separated from each other;

FIG. 8 is another sectional view illustrating the base and the biosensor being assembled;

FIG. 9 is a partly exploded perspective view illustrating a second embodiment of the physiological signal monitoring device according to the disclosure;

FIG. 10 is another partly exploded perspective view illustrating the second embodiment;

FIG. 11 is a sectional view illustrating a transmitter of the second embodiment at an initial position;

FIG. 12 is a sectional view illustrating the transmitter at an assembled position;

FIG. 13 is a schematic view illustrating an operation for separating a base and a transmitter from each other by at least one tool;

FIG. 14 is a sectional view illustrating a third embodiment of the physiological signal monitoring device according to the disclosure;

FIG. 15 is a schematic view illustrating an operation for separating a base and a transmitter of a fourth embodiment of the physiological signal monitoring device according to the disclosure, where the base is bent by applying a force on a side thereof;

FIG. 16 is a schematic view illustrating another operation for separating the base and the transmitter of the fourth embodiment, where the base is bent by applying a force on a corner thereof;

FIG. 17 is a perspective view illustrating a fifth embodiment of the physiological signal monitoring device according to the disclosure;

FIG. 18 is a partly exploded perspective view illustrating a sixth embodiment of the physiological signal monitoring device according to the disclosure;

FIG. 19 is an exploded perspective view illustrating a conventional physiological signal monitoring device

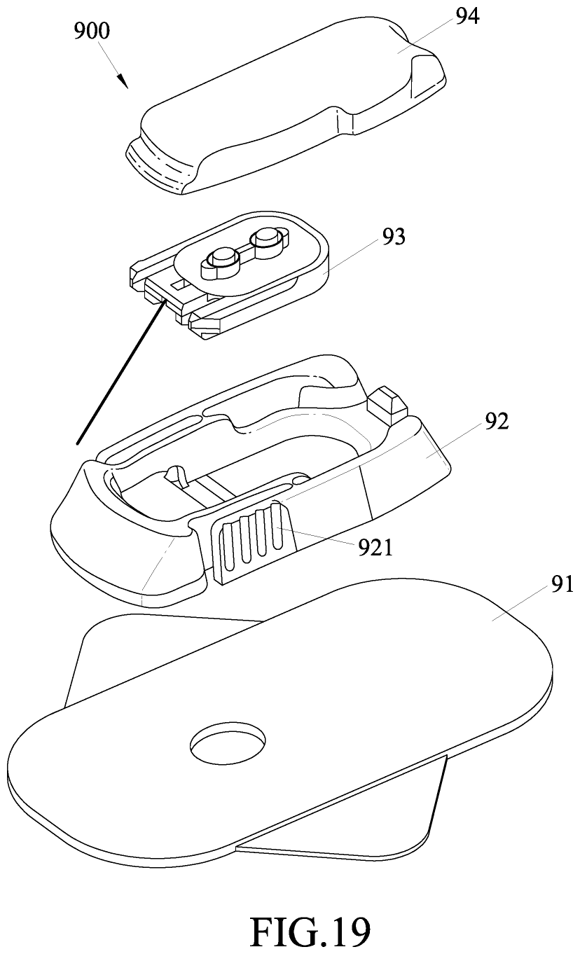

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

In the description of the disclosure, the terms “up”, “down”, “top”, “bottom” are meant to indicate relative position between the elements of the disclosure, and are not meant to indicate the actual position of each of the elements in actual implementations.

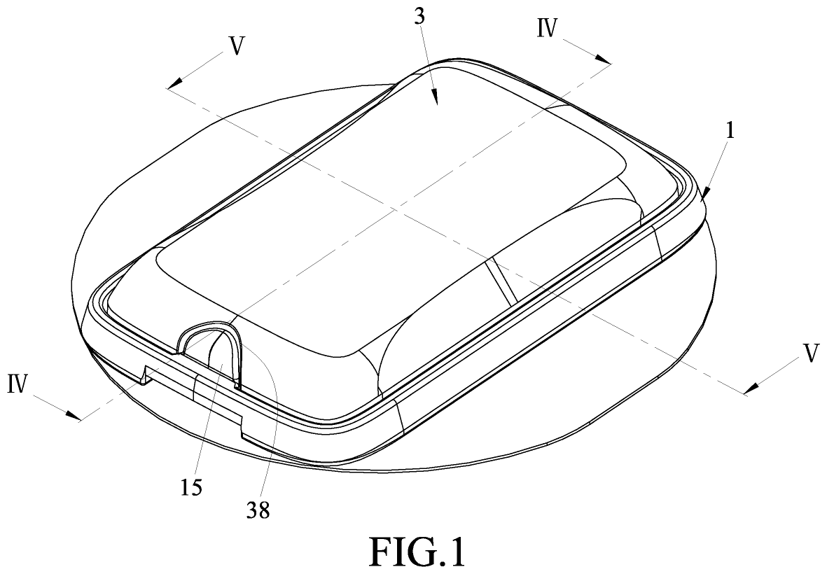

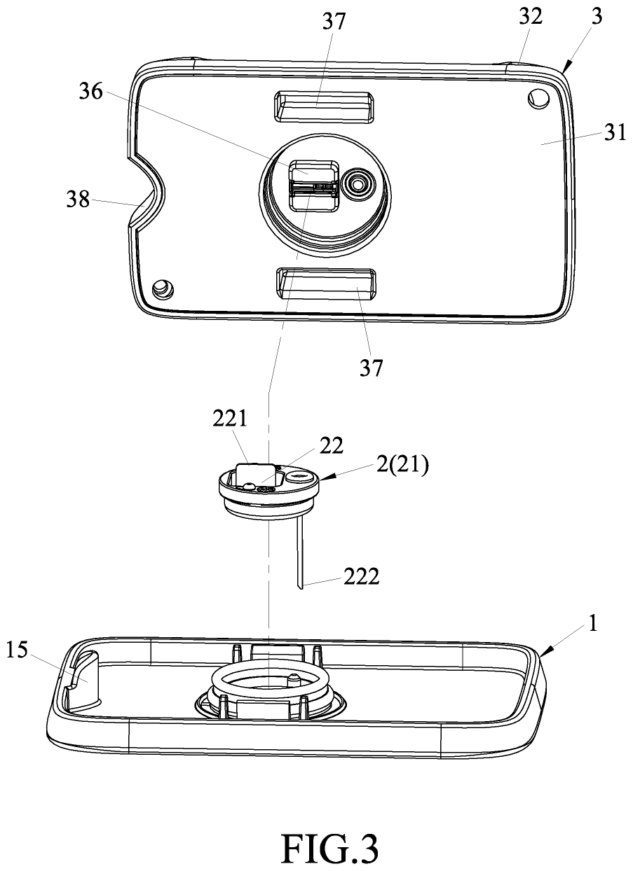

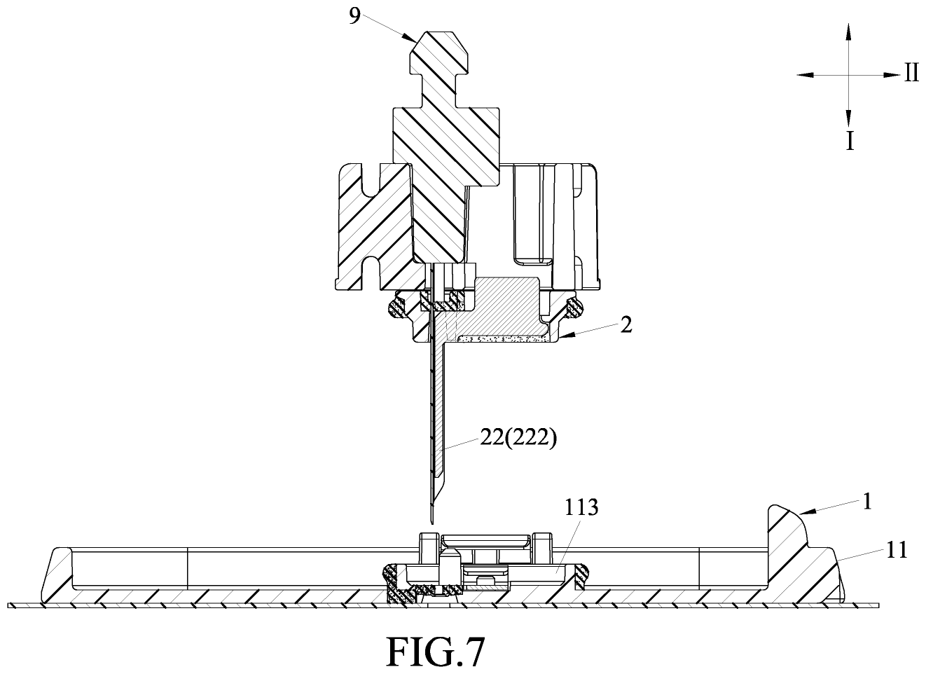

Referring to FIGS. 1 to 3, a first embodiment of a physiological signal monitoring device according to the disclosure is adapted to be mounted to a skin surface of a host (not shown) via an insertion tool 9 (see FIG. 7) of an insertion device (not shown) for measuring at least one analyte of the host and for transmitting a corresponding physiological signal corresponding to the analyte. In this embodiment, the physiological signal monitoring device is for measuring the glucose concentration in the interstitial fluid (ISF) of the host, but is not restricted to such.

The physiological signal monitoring device includes a base 1, a biosensor 2 that is mounted to the base 1 for generating a physiological signal, and a transmitter 3 that is removably mounted to the base 1 for receiving and transmitting the physiological signal.

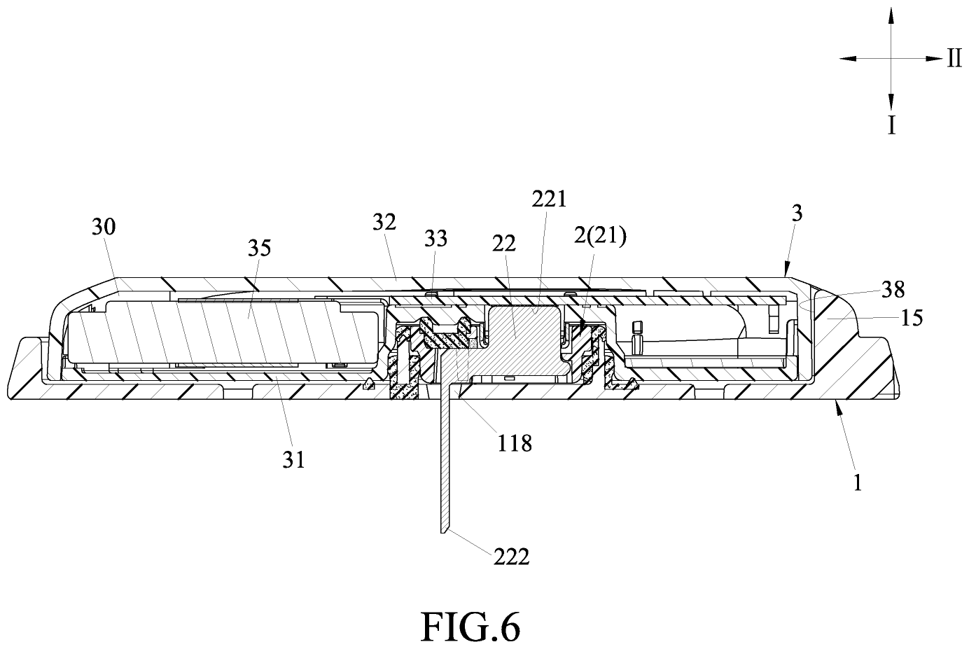

The base 1 includes a base body 11, and a pair of first alignment structures 12 that are disposed on the base body 11. The base body 11 has a bottom plate 111 adapted to be mounted to the skin surface of the host and perpendicular to a direction of a first axis an outer surrounding wall 112 that extends upwardly along the direction of the first axis (I) from a periphery of the bottom plate 111, and an inner surrounding wall 114 that protrudes from a top surface 115 of the bottom plate 111 and that cooperates with the bottom plate 111 to define a mounting groove 113. The bottom plate 111 has the top surface 115, a bottom surface 116 opposite to the top surface 115 in the direction of the first axis (I), and a through hole 118 (see FIG. 6) extending through the top and bottom surfaces 115, 116 of the bottom plate 111 and communicated to the mounting groove 113. The first alignment structures 12 are spaced apart from each other in a direction of a third axis (III), which is perpendicular to the first axis (I). A second axis (II), which be referenced herein, is perpendicular to both the first and third axes (I, III). In some embodiments, an angle between every two axes of the first second and third axes II, III) is not limited to 90 degrees. In addition, similarly, various axes to be disclosed herein, while defined to be perpendicular to one another in the disclosure, may not be necessarily perpendicular in actual implementation.

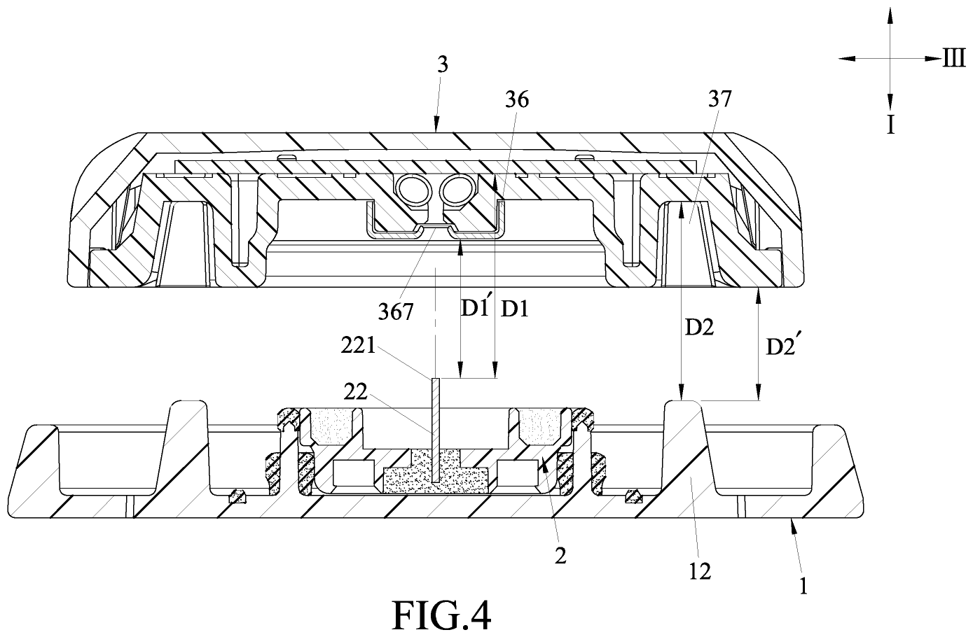

The first alignment structures 12 are disposed on a top portion of the base 1. In one embodiment the first alignment structures 12 are disposed adjacent to the biosensor 2, Specifically, the first alignment structures 12 protrude from the top surface 115 of the bottom plate ill of the base body 11. Each of the first alignment structures 12 includes an alignment block 121 and two limiting plates 122 that are respectively disposed at two opposite sides of the alignment block 121. The first alignment structures 12 are respectively disposed at two opposite sides of the mounting groove 113 in the direction of the third axis (III). In this embodiment, the alignment block 121 of each of the first alignment structures 12 is configured to be plate-shaped. In some embodiment, the alignment block 121 of each of the first alignment structures 12 may be frusto-pyramidal (see FIG. 4), preferably may be a frustum of a rectangular pyramid, and tapers away from the bottom plate 111 of the base body 11, but is not limited to such. In addition, the quantity of the first alignment structures 12 is not limited to two as mentioned in this embodiment. That is, the base 1 can have only one first alignment structure disposed adjacent to the biosensor 2 so as to achieve the same purpose.

In one embodiment, the base 1 may include a third alignment structure 15 that is disposed at the periphery of the base body 11. Specifically, the third alignment structure 15 is disposed at the outer surrounding wall 112. More specifically, the third alignment structure 15 extends from the outer surrounding wall 112 toward the mounting groove 113, and is a tab.

Referring back to FIGS. 1 to 3, the biosensor 2 includes a mounting seat 21 that is mounted to the mounting groove 113 of the base body 11, and a sensing strip 22 that is carried and limited by the mounting seat 21, and that is adapted for measuring the at least one analyte of the host and for sending the corresponding physiological signal to the transmitter 3.

The sensing strip 22 has a sensing section 222 and a signal output section 221. The sensing section 222 is adapted to be inserted underneath the skin surface of the host for measuring the physiological signal corresponding to at least one physiological parameter of the at least one analyte of the host. The signal output section 221 protrudes from the top surface 115 of the bottom plate all of the base body 11 for transmitting the physiological signal. In this embodiment, the mounting seat 2i is made independently before being mounted to the base 1. In some embodiment, the mounting seat 21 and the base 1 may be formed as one piece. The first alignment structures 12 are disposed adjacent to the biosensor 2, and are spaced apart from the biosensor 2 in the direction of the third axis (III).

Referring to FIG. 6, the transmitter 3 includes a bottom portion 31 that adjacent to the base body 11 and that faces the top surface 115 of the bottom plate 111, a top portion 32 that cooperates with the bottom portion 31 to define an inner space 30 therebetween, a circuit hoard 33 that is disposed in the inner space 30, a battery 35 that is disposed in the inner space 30 and that is electrically connected to the circuit board 33, a connection port 36 that is connected to a bottom surface of the circuit board 33 and that extends outwardly from the inner space 30 toward the base body 11, and a pair of second alignment structures 37 that are disposed at the bottom portion 31 and that respectively correspond in position to the first alignment structures 12 of the base 1. In one embodiment, each of the second alignment structures 37 is configured as a groove that is indented from a bottom portion of the transmitter 3, and that corresponds in shape to the respective one of the first alignment structures 12. That is to say, when the first alignment structures are configured to be frusto-pyramidal, each of the second alignment structures 37 is configured as a frusto-pyramidal groove. The shapes of the first alignment structures 12 and the second alignment structures 37 are not limited to such. In one embodiment, the number of the second alignment structure 37 may be only one. In one embodiment, the transmitter 3 may include a fourth alignment structure 38 that is disposed at a periphery of the transmitter 3, and that corresponds in shape to the third alignment structure 15. Specifically, the fourth alignment structure 38 is disposed at a periphery of the transmitter 3 cooperatively formed by the bottom portion 31 and the top portion 32, and is configured as an arced recess, but is not limited to such.

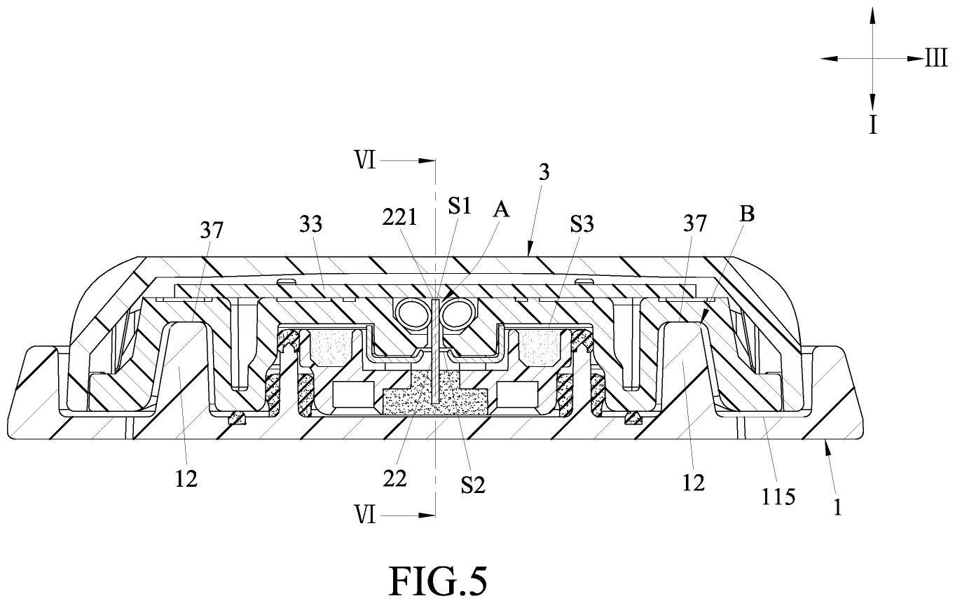

The transmitter 3 is selectively located at one of an initial position (see FIG. 4) and an assembled position (see FIGS. 5 and 6) relative to the base 1 in the direction of the first axis (I). The connection port 36 of the transmitter 3 has a connection part for connecting with the signal output section 221 of the sensing strip 22. In particular, the connection part of the transmitter 3 and the signal output section 221 of the sensing strip 22 are shaped to be complementary to each other, and cooperatively form a connection portion (A) after the transmitter 3 moves from the initial position to the assembled position by a first travel distance (D1) in the direction of the first axis M.

Each of the second alignment structures 37 and the respective one of the first alignment structures 12 are shaped to be complementary to each other, and cooperatively form an alignment portion (B) after the transmitter 3 moves from the initial position to the assembled position by a second travel distance (D2) in the direction of the first axis (I).

Preferably, the first travel distance (D1) is greater than or equal to the second travel distance (D2). By such, there would be a first gap (S1, see FIG. 5) between the biosensor 2 and the transmitter 3 that prevent the signal output section 221 of the sensing strip 22 and the connection part of the transmitter 3 from damage due to collision therebetween. Preferably, the connection part of the transmitter 3 has an insertion hole 367, and each of the second alignment structures 37 is configured as a groove formed at the bottom portion or the transmitter 3.

Preferably, at the initial position, a first distance (D1′) between a bottom of the connection part (i.e., an opening of the insertion hole 367) and the signal output section 221 of the sensing strip 22 is greater than a second distance (D2′) between a top of the first alignment structure 12 and the bottom of the transmitter 3. By such, collision between the signal output section 221 of the sensing strip 22 and the connection part of the transmitter 3 is prevented before the first alignment structures 12 start to be limited by the second alignment structures 37. A distance between the connection part and the top portion 32 of the transmitter 3 is smaller than a distance between an opening of the second alignment structure 37 and the top portion 32 of the transmitter 3.

Accordingly, during the movement of the transmitter 3 from the initial position to the assembled position, a top of each of the first alignment structures 12 will be limited by each of the second alignment structures 37 firstly to guide movement of the transmitter 3 with respect to the base 1 in the direction of the second axis (II) and/or the direction of the third axis (III) for aligning the signal output section 221 of the sensing strip 22 and the insertion hole 367 of the transmitter 3 with each other, so as to prevent the signal output section 221 from colliding the rigid portion of the connection port 36 that defines the insertion hole 367 By virtue of the limitation between the first alignment structures 12 and the second alignment structures 37, a second gap (S2′) between the signal output section 221 and the rigid portion of the connection port 36 that defines the insertion hole 367 is generated.

Preferably, taking an inner surface of the top portion 32 of the transmitter 3 (or the circuit board 33) as reference, the length of the alignment portion (B) in the direction of the first axis (I) is greater than that of the connection portion (A). As such, a third gap (S3) is generated during assembling the transmitter 3 and the base 1 to prevent the collision between the signal output section 221 of the sensing strip 22 and the bottom portion of the transmitter 3.

Referring to FIGS. 5 and 6, when the transmitter 3 is at the assembled position, the signal output section 221 of the sensing strip 22 is inserted into the connection port 36 through the insertion hole 367 to form the electric connection between the biosensor 2 and the transmitter 3.

In assembling the transmitter 3 onto the base 1, before the first alignment structures 12 are limited by the second alignment structures 37 (i.e., the first alignment structures 12 and the second alignment structures 37 are separated), during the limitation between the first alignment structures 12 and the second alignment structures 37 (i.e., the first alignment structures 12 and the second alignment structures 37 move relative to each other), and after the first alignment structures 12 cooperate with the second alignment structures 37 to form the alignment portions (B), at least one of the first gap (S1), the second gap (S2) and the third gap (S3) serves to prevent at least one of the signal output section 221 of the sensing strip 22 and the connection part from collision. That is to say, any one of the first gap (S1), the second gap (S2) and the third gap (S3) can be defined as a safety gap.

In the first embodiment, the relative movement between the first alignment structures 12 and the second alignment structures 37 as the first alignment structures 12 are limited by the second alignment structures 37 guides movement between the transmitter 3 and the base 1 in at least one of the directions of the second axis (II) and the third axis (III) for aligning the signal output section 221 of the sensing strip 22 and the insertion hole 367 of the transmitter 3 with each other. Referring to FIGS. 5 and 6, when the transmitter 3 is at the assembled position, the first alignment structures 12 and the second alignment structures 37 are engaged with each other, and the third alignment structure 15 and the fourth alignment structure 38 are engaged with each other (with reference FIG. 1). By virtue of the third alignment structure 15 and the fourth alignment structure 38, and by virtue of the first alignment structures 12 and the second alignment structures 37 that guide movement between the bottom portion 31 of the transmitter 3 and the base 1 in at least one of the directions of the second axis (II) and the third axis (III), when the bottom portion 31 of the transmitter 3 is assembled to the base 1 in the direction of the first axis (I), the signal output section 221 of the sensing strip 22 and the connection part of the transmitter 3 are ensured to be aligned with each other, and misalignment between the signal output section 221 of the sensing strip 22 and the connection part of the transmitter 3 due to manufacturing tolerances can be prevented. When the transmitter 3 is at the assembled position, movement of the transmitter 3 in the direction of the second axis (II) and/or the direction of the third axis (III) is limited by the outer surrounding wall 112 of the base 1.

A method for mounting the physiological signal monitoring device onto the skin surface of the host includes steps of:

1) providing the physiological signal monitoring device in the above;

2) executing a biosensor-mounting process, in which the biosensor 2 is mounted to a top portion of the base 1;

3) executing a first transmitter mounting process, in which the transmitter 3 is moved relative to the base 1 in the direction of the first axis (I) from the initial position toward the assembled position with the bottom portion of the transmitter 3 facing the top portion of the base 1, and the safety gap prevent the signal output section 221 of the sensing strip 22 or the connection part from collision;

4) executing an alignment process, in which the relative movement between the first alignment structures 12 and the second alignment structures 37 when the first alignment structures 12 are limited by the second alignment structures 37 guides movement between the transmitter 3 and the base 1 in at least one of the directions of the second axis (II) and the third axis (III) for aligning the signal output section 221 of the sensing strip 22 and the connection part of the transmitter 3 with each other, the outer surrounding wall 112 extending from the periphery of the bettors plate 111 of the base 1 guides movement of the transmitter 3 relative to the base body 11 in the direction of the second axis (II) and/or the direction of the third axis (III), and the third alignment structure 15 of the base 1 and the fourth alignment structure 38 of the transmitter guides movement of the transmitter 3 relative to the base body 11 in the direction of the second axis (Ii) and/or the direction of the third axis (III) to align the transmitter 3 and the base 1 with each other; and

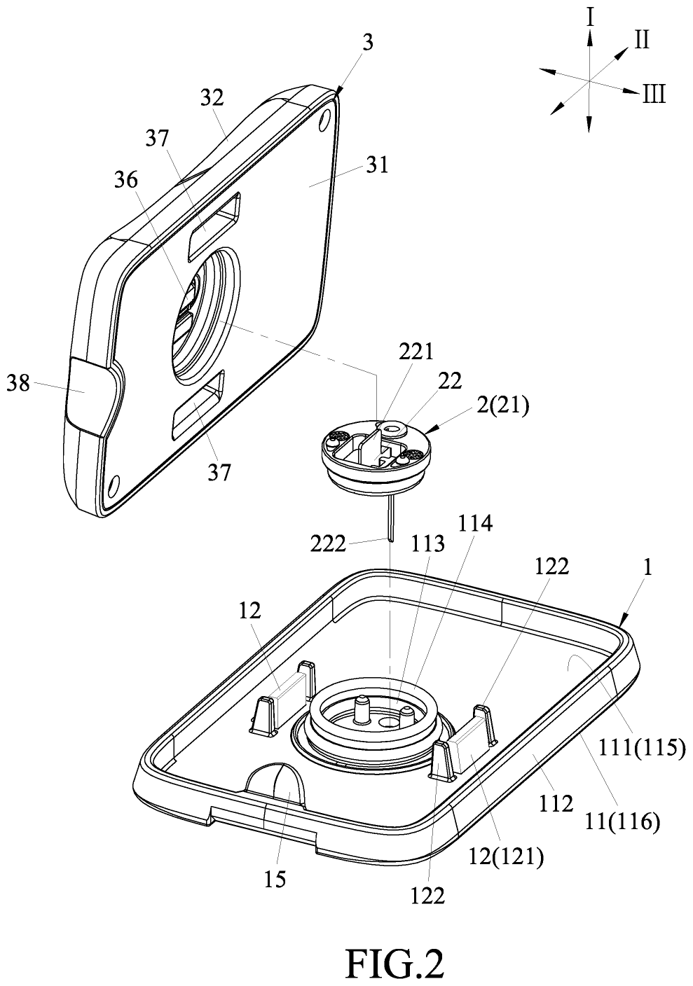

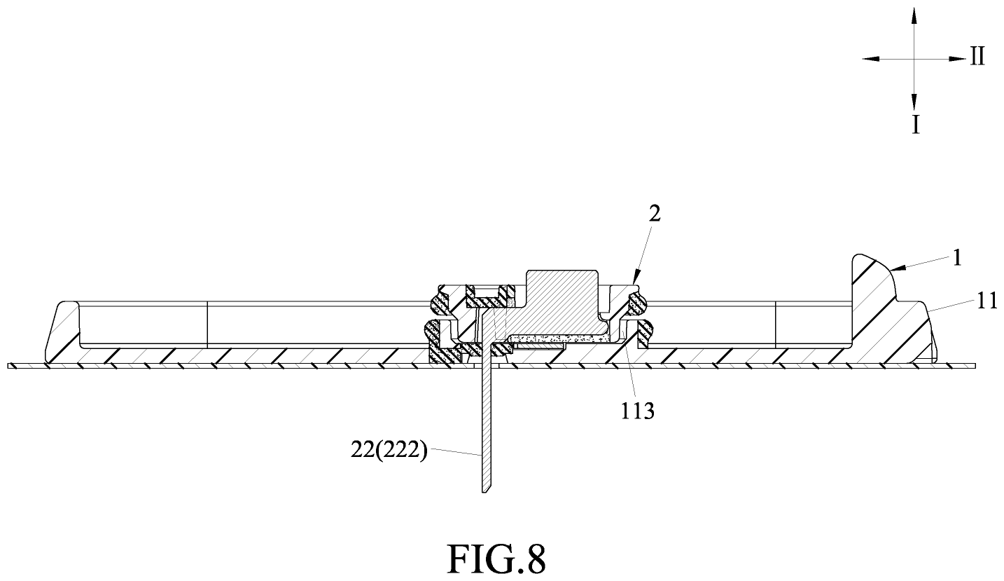

5) executing a second transmitter mounting process, in which the transmitter 3 is moved relative to the base 1 in the direction of the first axis (1) to be mounted onto the base 1, and the signal output section 221 of the sensing strip 22 is connected to the connection part to form the electric connection between the biosensor 2 and the transmitter 3 Referring to FIG. 2, the base 1, the biosensor 2, and the transmitter 3 are separated from one another before use, and are coupled to one another to be mounted to the skin surface of the host. Referring to FIGS. 7 and 8, during the assembling, the base 1 and the biosensor 2 are coupled to the insertion tool 9 of the insertion device, and the base body 11 is attached to the skin surface via an adhesive pad 16. Then, as the sensing section 222 of the sensing strip 22 is carried by the insertion tool 9 to extend through the through hole 118 of base body and subsequently inserted underneath the skin surface of the host, the mounting seat 21 of the biosensor 2 is mounted to the mounting groove 113 of the base body 11. After the sensing section 222 of the sensing strip 22 is inserted underneath the skin surface of the host, the insertion tool 9 is drawn out from the host so that the insertion device is separated from the base 1 and the biosensor 2, while the base 1 and the biosensor 2 remain coupled to one another. Lastly, to finish the assembling, the transmitter 3 covers to the base body 11 so that the first and second alignment structures 12, 37 are driven by an external force to be engaged with each other, while the signal output section 221 of the sensing strip 22 is inserted into the connection port 36 via the insertion hole 367 in the direction of the first axis (D1). After the physiological signal monitoring device is assembled, the sensing strip 22 of the biosensor 2 is able to measure the at least one analyte of the host and send the corresponding physiological signal to an external receiving device (not shown) via the transmitter 3.

To be environmentally friendly, some components of the physiological signal monitoring device according to the disclosure may be reusable. In one embodiment, the transmitter 3 is reusable. When the biosensor 2 needs to be replaced, a user may separate the transmitter 3 from the used biosensor 2 and base 1, and mount the transmitter 3, a new biosensor 2 and a new base 1 onto the skin surface of the host via the mounting method above. In one embodiment, the service life of the biosensor 2 is about two weeks, but may be different in other embodiments according to the actual use, the materials of the components or different kinds of components.

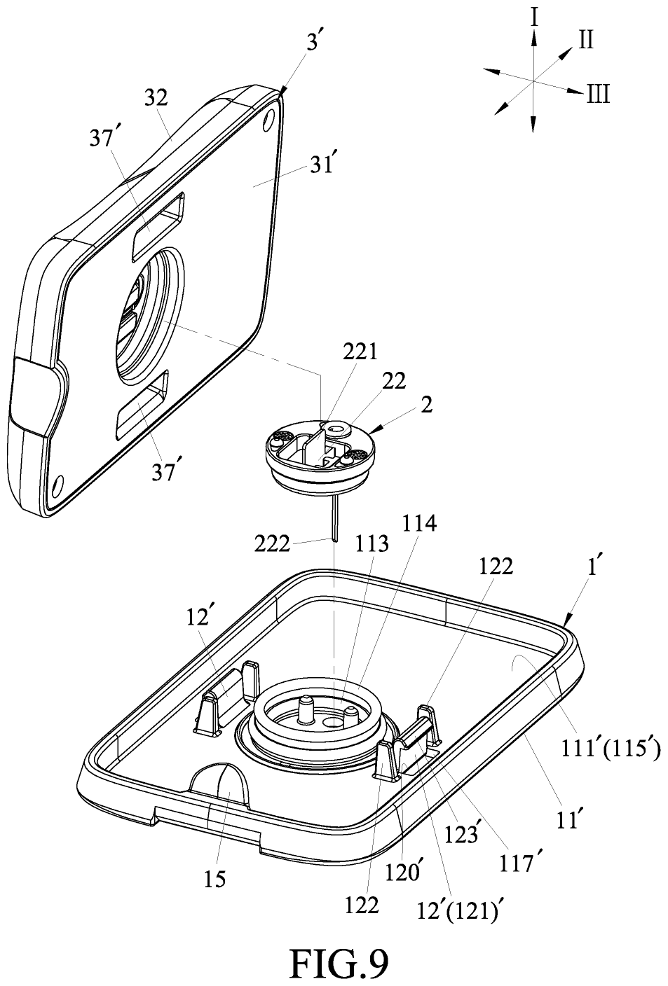

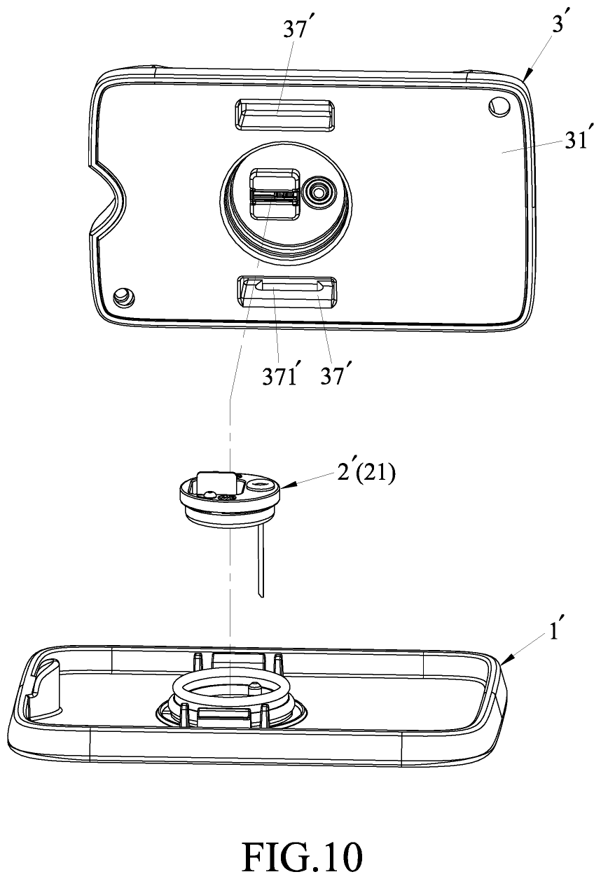

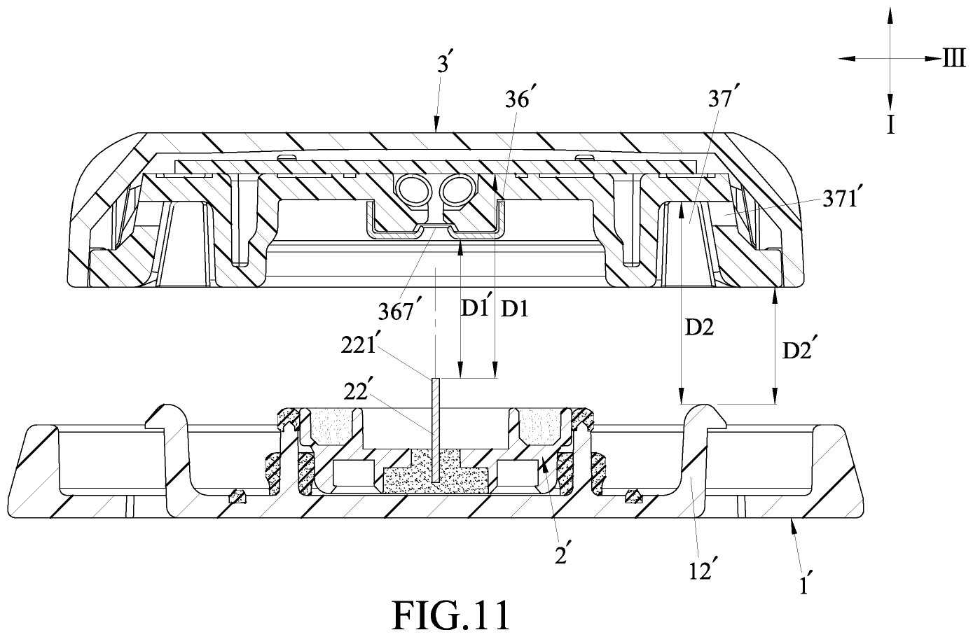

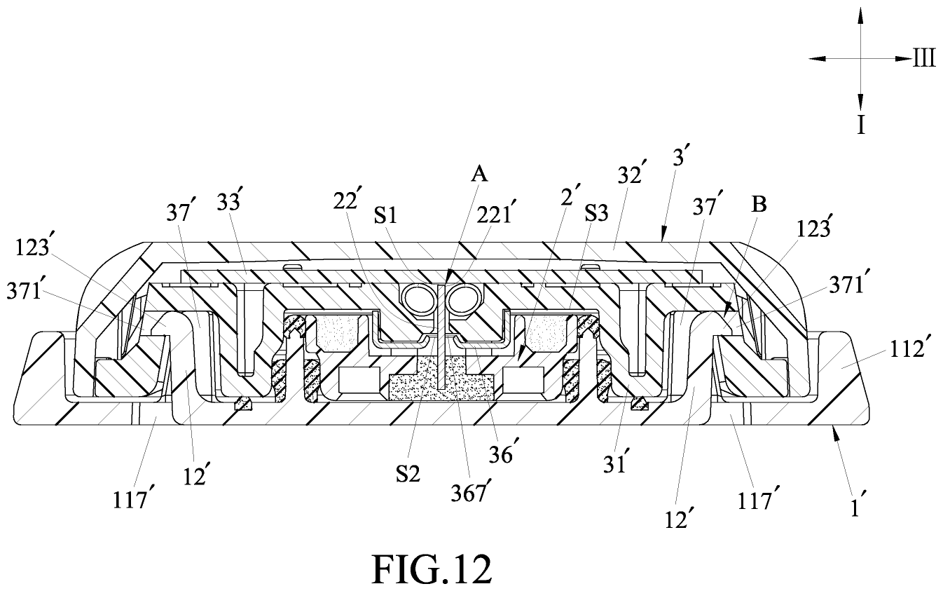

Referring to FIGS. 9 to 12, a second embodiment of the physiological signal monitoring device according to the disclosure is similar to the first embodiment with the following differences. Each of the first alignment structures 12′ of the base 1′ includes an alignment block 121′ at the top side of the base 1′. Each of the alignment blocks 121′ has a base part 120′ disposed on the top surface 115′ of the bottom plate 111′, and a first engaging part 123′ that is connected to an end of the base part 120′ distal from the bottom plate 111′. The first engaging part 123′ of each of the first alignment structures 12′ extends away from another one of the first engaging parts 123′. That is, the first engaging part 123′ extends toward the outer edge of the base body 11′ (see FIGS. 11 and 12), and is configured as a hook. Each of the second alignment structures 37′ includes a second engaging part 371′ that corresponds to the first engaging part 123′ of a respective one of the first alignment structures 12′. When the transmitter 3′ is at the assembled position, the first engaging part 123′ of each of the first alignment structures 12′ and the second engaging part 371′ of the respectively one of the second alignment structures 37′ are engaged with each other. In one embodiment, the second engaging part 371′ may be a recess that is formed in an inner surface of the second alignment structure 37′. When the transmitter 3′ is at the assembled position, the first engaging part 123′ of each of the first alignment structures 12′ at least partially engages the second engaging part 371′ of the respectively one of the second alignment structures 37′. The base body 11′ and the first alignment structures 12′ may be formed as one piece through an injection molding technique, the engaging direction of the first engaging parts 123′ of the first alignment structures 12′ (in direction(s) transverse to the first axis (I)) may enhance the concentricity of the inner structure of the base 1. In addition, since the second engaging parts 371′ are recesses that are respectively formed in inner surfaces of the second alignment structures 37′, and since the first engaging parts 123′ of the first alignment structures 12′ engages the second engaging parts 371′ in the directions toward the outer edge of the base body 11′, the first alignment structures 12′ are limited by a side wall of the bottom portion 31′ of the transmitter 3′, so as to enhance the stability of the engagement between the first alignment structures 12′ and the second alignment structures 37′. In one embodiment, the first engaging part 123′ of each of the first alignment structures 12′ may extend toward another one of the first alignment structures 12′, but is not limited to such.

According to the above, the transmitter 3′ and the base 1′ can be assembled easily. Since the first alignment structures 12′ are disposed on the top surface 115′ of the base body 11 and since the second alignment structures 37′ are disposed at the bottom portion 31′ of the transmitter 3′, the components in the transmitter 3′ may not be damaged during the engaging process between the first alignment structures 12′ and the second alignment structures 37′. It should be noted that, in the second embodiment, the first alignment structures 12′ am the second alignment structures 37′ (and the first engaging part 123′ and second engaging part 371′) are disposed to be distal from a periphery of the physiological signal monitoring device cooperatively formed by the base 1′ and the transmitter 3′, the periphery of the physiological signal monitoring device needs not be provided with engaging or detaching means, and is uninterrupted, so as to facilitate miniaturization of the physiological signal monitoring device.

Referring to FIG. 11, at the initial position, a first distance (D1′) between a bottom of the connection part (i.e., an opening of the insertion hole 367′) and the signal output section 221′ of the sensing strip 22′ is greater than a second distance (D2′) between the top of the first alignment structure 12′, and the bottom of the transmitter 3′.

Accordingly, during the movement of the transmitter 3′ from the initial position to the assembled position, the first alignment structures 12′ are limited by the second alignment structures 37′ firstly to guide movement between the transmitter 3′ and the base 1′ in the direction of the second axis (II) and/or the direction of the third axis (III) for aligning the signal output section 221′ of the sensing strip 22′ and the insertion hole 367′ of the transmitter 3′ with each other, so as to prevent the signal output section 221′ from colliding the rigid portion of the connection port 36′ that defines the insertion hole 367′. A second gap (S2′) between the signal output section 221′ and the rigid portion of the connection port 36′ that defines the insertion hole 367′ is generated.

Referring to FIG. 12, when the transmitter 3′ is at the assembled position, the signal output section 221′ of the sensing strip 22′ is inserted into the connection port 36′ through the insertion hole 367′ to form the electric connection between the biosensor 2′ and the transmitter 3′.

Referring to FIGS. 11 and 12, in the engaging process as the first alignment structures 12′ are limited by the second alignment structures 37′, the second gap (32) formed between the signal output section 221′ and the rigid portion of the connection port 36′ that defines the insertion hole 367′ prevents at least one of the signal output section 221′ and the rigid portion of the connection port 36′ from collision, and the first gap (S1) also prevents at least one of the signal output section 221′ and the connection part from collision. When the transmitter 3′ is at the assembled position, movement of the transmitter 3′ in the direction of the second axis (II) and/or the direction of the third axis (III) is limited by the outer surrounding wall 112′ of the base 1′. Taking an inner surface of the top portion 32′ of the transmitter 3′ (or the circuit board 33′) as reference, the length of the alignment portion (B) in the direction of the first axis (I) is greater than that of the connection portion (A). As such, a third gap (33) is generated during assembling the transmitter 3′ and the base 1′.

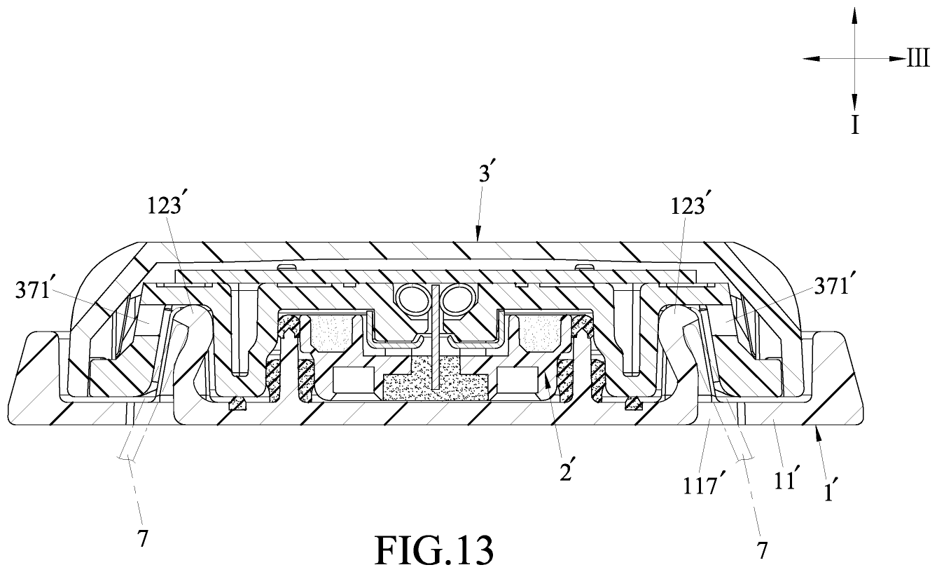

Referring back to FIG. 9, the base body 11′ further includes two openings 117′ that are formed through the base body 11′, that are spaced apart from each other in the direction of the third axis (III) and that respectively correspond in position to the first engaging part 123′ of the first alignment structures 12′. To replace the base 1′ and the biosensor 2′, the base 1′ may be firstly removed from the skin surface of the host. Then, with reference to FIG. 13, a user may use his/her fingers or other disassembly tools 7 to apply an external force through the openings 117′ to push against the first alignment structures 12′, the second alignment structures 37′, or a location where the first and second alignment structures 12′, 37′ couple to each other to separate the first alignment structures 12′ and the second alignment structures 37′ from each other, so as to separate the transmitter 3′ from the base 1′ and the biosensor 2′.

The second embodiment has functions the same as those of the first embodiment.

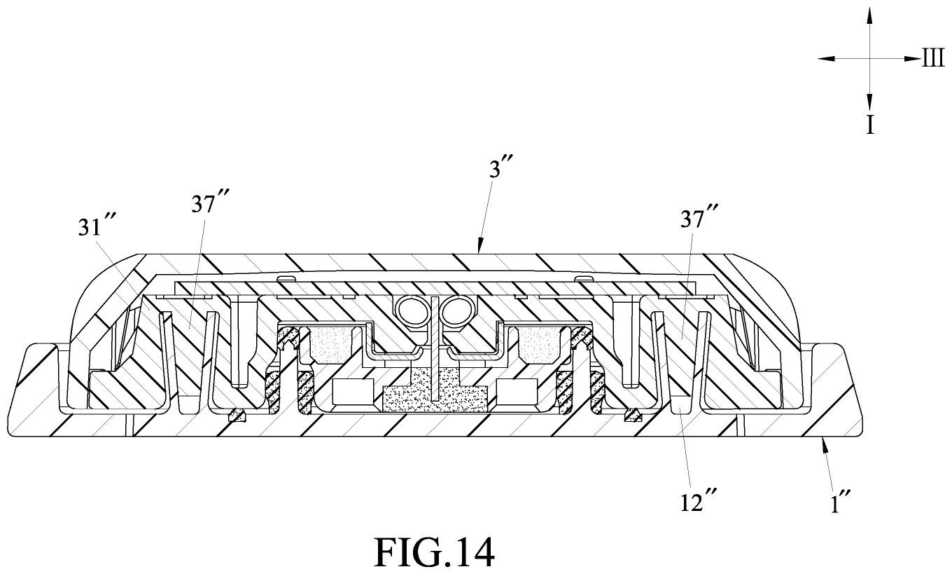

Referring to FIG. 14, a third embodiment of the physiological signal monitoring device according to the disclosure is similar to the first embodiment. Each of the first alignment structures 12″ and the respective one of the second alignment structures 37″ are shaped to be complementary to each other. In this embodiment, each of the first alignment structures 12″ is configured as a groove formed in the base 1″, and each of the second alignment structures 37″ is configured as a protrusion disposed on the bottom portion 31″ of the transmitter 3″. Specifically, each of the second alignment structures 37″ is frusto-conical, and each of the first alignment structures 12″ is configured as a frusto-conical groove. The third embodiment has functions the same as those of the first and second embodiments.

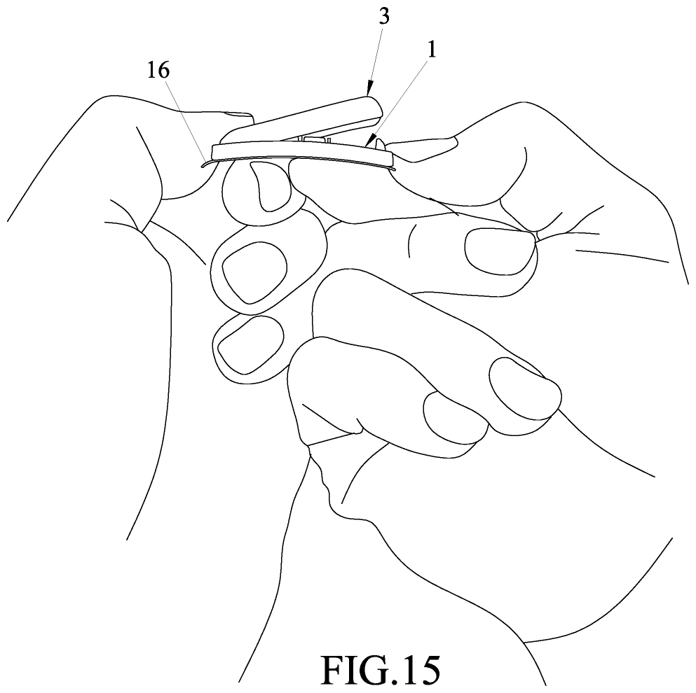

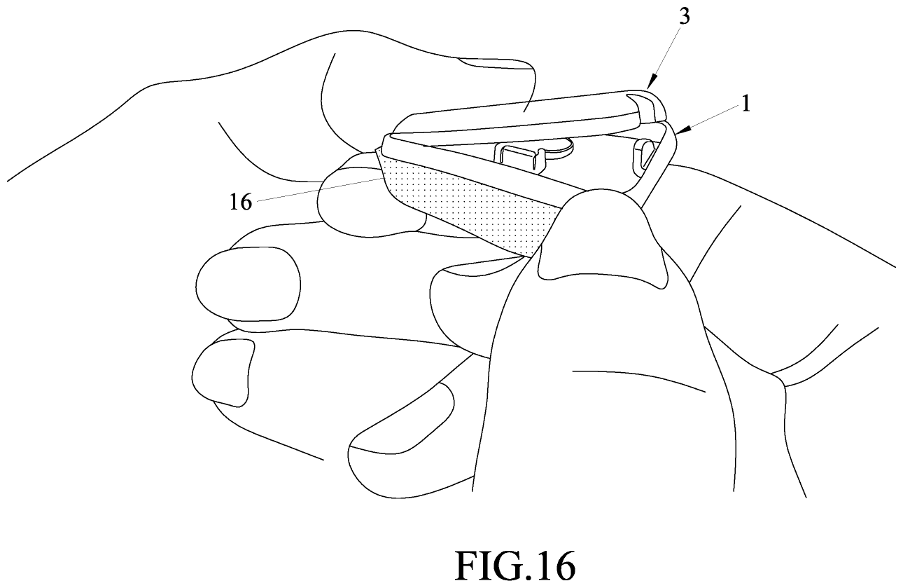

Referring to FIGS. 15 and 16, a fourth embodiment of the physiological signal monitoring device according to the disclosure as similar to the first embodiment with the following differences.

The base 1 is flexible, such that, by applying an external force to bend the base 1 on a side thereof (see FIG. 15) or on a corner thereof (FIG. 16), the transmitter 3 is permitted to be separated from the base 1.

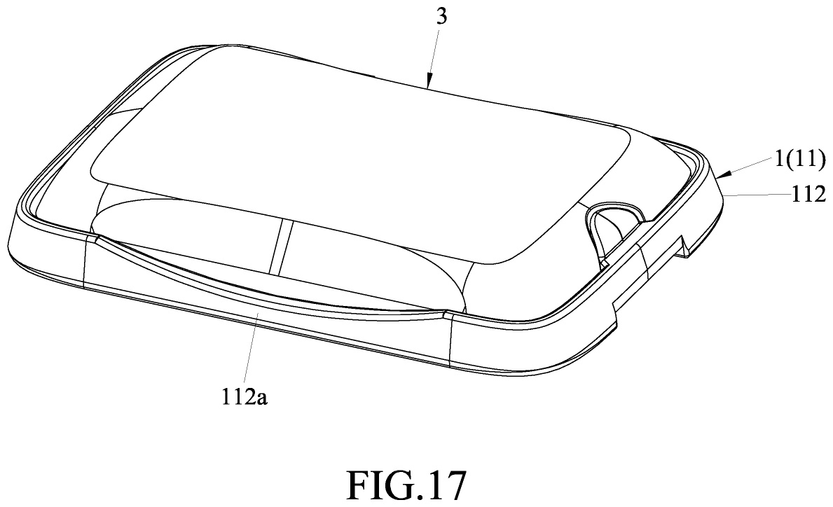

FIG. 17 illustrates a fifth embodiment of the physiological signal monitoring device according to the disclosure. The outer surrounding wall 112 of this embodiment has two short portions 112a respectively disposed at two longer sides thereof. Top edges of the short portions 112a are arc-shaped such that the height of each of the short portions 112a with respect to the top surface 115 (see FIG. 2) is ununiform. The outer surrounding wall 112 has a second height at centers of the short portions 112a. As such, the base body 11 can be easily bent at the centers of the short portions 112a.

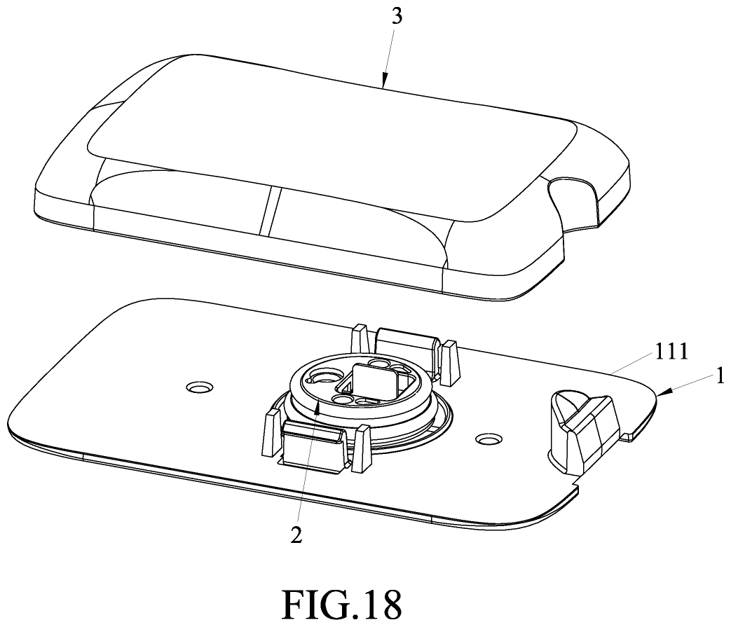

FIG. 18 illustrates a sixth embodiment of the physiological signal monitoring device according to the disclosure. The outer surrounding wall 112 of the base body 11 is omitted, and the top portion 32 of the transmitter 3 extends downwardly to surround a periphery of the bottom plate 111 of the base body 11.

In summary, by virtue the first alignment structures 12, 12′, 12″ of the base 1, 1′, 1″ and the second alignment structures 37, 37′, 37″ of the transmitter 3, 3′, 3″, the signal output section 221, 221′, 221″ of the sensing strip 22, 22′, 22″ and the connection part of the transmitter 3, 3′, 3″ are prevent from collision in the assembling process between the first alignment structures 12, 12′, 12″ and the second alignment structures 37, 37′, 37″.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description. thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A physiological signal monitoring device adapted to be mounted to a skin surface of a host and to be partially inserted underneath the skin surface for measuring at least one analyte of the host, comprising:
a base including at least one first alignment structure that is disposed on a top portion of the base;
a biosensor including
a sensing section that is adapted to be inserted underneath the skin surface for measuring the at least one analyte, and
a signal output section that is disposed at the top portion of the base; and
a transmitter selectively located at one of an initial position and an assembled position relative to the base in the direction of a first axis, the transmitter including
connection part disposed at a bottom portion of the transmitter, the connection part and the signal output section of the biosensor being shaped to be complementary to each other, and cooperatively forming a connection portion after the transmitter moving from the initial position to the assembled position by a first travel distance in the direction of the first axis, and
at least one second alignment structure that is disposed at the bottom portion of the transmitter and that correspond in position to the first alignment structure of the base, the second alignment structure and the first alignment structure being shaped to be complementary to each other, and cooperatively forming an alignment portion after the transmitter moving from the initial position to the assembled position by a second travel distance in the direction of the first axis;
wherein, the first travel distance is greater than or equal to the second travel distance, and a safety gap is formed between the transmitter and the biosensor;
wherein, when the transmitter moves from the initial position toward the assembled position, the safety gap serves to prevent at least one of the connection part of the transmitter and the signal output section of the biosensor from collision.

2. The physiological signal monitoring device as claimed in claim 1, wherein when the transmitter is at the initial position, a distance between the first alignment structure and the bottom portion of the transmitter in the direction of the first axis is smaller than or equal to a distance between the bottom portion of the transmitter and the signal output section of the biosensor in the direction of the first axis.

3. The physiological signal monitoring device as claimed in claim 1, wherein the signal output section of the biosensor protrudes from the top portion of the base, the connection part of the transmitter having an insertion hole, the signal output section of the biosensor being inserted into the transmitter through the connection part to form an electric connection between the biosensor and the transmitter when the transmitter is at the assembled position.

4. The physiological signal monitoring device as claimed in claim 1, wherein the biosensor includes
a mounting seat, and
a sensing strip that is carried by the mounting seat and that has the sensing section and the signal output section, the sensing section being adapted to be inserted underneath the skin surface of the host for measuring a physiological signal corresponding to the at least one analyte of the host, the signal output section being for transmitting the physiological signal, the mounting seat and the base being formed as one piece.

5. The physiological signal monitoring device as claimed in claim 1, wherein the at least one first alignment structure includes two first alignment structures, the first alignment structures are disposed adjacent to the biosensor, and are spaced apart from each other in a direction perpendicular to the first axis.

6. The physiological signal monitoring device as claimed in claim 1, wherein the first alignment structure protrudes from the top portion of the base, the second alignment structure being configured as a groove that is indented from the bottom portion of the transmitter, relative movement between the first alignment structure and the second alignment structure as the first alignment structure is limited by the second alignment structure guiding movement of the transmitter with respect to the base in at least one of the directions of a second axis and a third axis that are different from the first axis for aligning the signal output section of the biosensor and the connection part of the transmitter with each other.

7. The physiological signal monitoring device as claimed in claim 6, wherein the transmitter further includes a top portion that cooperates with the bottom portion to define an inner space therebetween, a distance between the connection part and the top portion of the transmitter is smaller than a distance between an opening of the second alignment structure and the top portion of the transmitter.

8. The physiological signal monitoring device claimed in claim 6, wherein the first axis is perpendicular to the second axis, and is perpendicular to the third axis.

9. The physiological signal monitoring device as claimed in claim 1, wherein the first alignment structure of the base includes a first engaging part, and the second alignment structure of the transmitter includes a second engaging part, the first engaging part of the first alignment structure and the second engaging part of the second alignment structure being engaged with each other when the transmitter is at the assembled position.

10. The physiological signal monitoring device as claimed in claim 9, wherein the first alignment structure protrudes from top portion of the base, the second alignment structure being configured as a groove that is indented from the bottom portion of the transmitter, the second engaging part of the second alignment structure being a recess that is formed in an inner surface of the second alignment structure, the first engaging part of the first alignment structure at least partially engaging the second engaging part of the second alignment structure when the transmitter is at the assembled position.

11. The physiological signal monitoring device as claimed in claim 9, wherein the base further includes at least one opening that is formed through the base and that correspond in position to the first engaging part of the first alignment structure, an external force being permitted to be applied through the opening to separate the first engaging part of the first alignment structure and the second engaging part of the second alignment structure from each other so as to separate the transmitter from the base.

12. The physiological signal monitoring device as claimed in claim 1, wherein the base further includes a bottom plate that is adapted to be mounted to the skin surface of the host, and an outer surrounding wall that extends upwardly from a periphery of the bottom plate, the transmitter being limited by the outer surrounding wall when the transmitter is at the assembled position.

13. The physiological signal monitoring device as claimed in claim 1, wherein the base further includes a third alignment structure that is disposed at the periphery of the base, and the transmitter further includes a fourth alignment structure that is disposed at a periphery of the transmitter, and that corresponds in shape to the third alignment structure.

14. A method for mounting a physiological signal monitoring device onto the skin surface of the host comprising steps of:
a) providing the physiological signal monitoring device of claim 1;
b) executing a first transmitter mounting process, in which the transmitter is moved relative to the base in the direction of the first axis from the initial position toward the assembled position with the bottom portion of the transmitter facing the top portion of the base, and the safety gap prevent at least one of the signal output section of the biosensor and the connection part from collision;
c) executing an alignment process, in which the relative movement between the first alignment structure and the second alignment structure as the first alignment structure and the second alignment structure are limited with each other guides movement between the transmitter and the base for aligning the signal output section of the biosensor and the connection part of the transmitter with each other; and
d) executing a second transmitter mounting process, in which the transmitter is moved relative to the base in the direction of the first axis to be mounted onto the base, and the signal output section of the biosensor is connected to the connection part of the transmitter to form an electric connection between the biosensor and the transmitter.

15. The method of claim 14, further comprising a step of:
e) executing a biosensor-mounting process, in which the biosensor is mounted to the top portion of the base.

16. The method of claim 14, the signal output section of the biosensor protruding from the top portion of the base, the connection part of the transmitter having an insertion hole, the method further comprising a step of:
f) inserting the signal output section of the biosensor protruding into the transmitter via the connection part.

17. The method of claim 14, the first alignment structure protruding from the top portion of the base, the second alignment structure being configured as a groove that is indented from the bottom portion of the transmitter, step c) further comprising a sub-step of:
g) guiding the transmitter relative to the base in at least one of the directions of a second axis and a third axis that are different from the direction of first axis.

18. The method of claim 14, the first alignment structure having a first engaging part, the second alignment structure having a second engaging part, step d) further comprising a sub-step of:
h) engaging the first engaging part of the first alignment structure with the second engaging part of the second alignment structure to assemble the transmitter onto the base.

19. The method of claim 14, the base further includes an outer surrounding wall that extends upwardly from a bottom plate thereof, step c) further comprising a sub-step of:
i) guiding the transmitter relative to the base in at least one of the directions of a second axis and a third axis that are different from the direction of first axis.

20. The method of claim 14, the base further including a third alignment structure that is disposed at the periphery or the base, the transmitter further including a fourth alignment structure that is disposed at a periphery of the transmitter and that corresponds in shape to the third alignment structure, step c) further comprising a sub-step of:
j) guiding the transmitter relative to the base in at least one of the directions of a second axis and a third axis that are different from the direction of first axis.
